(19) United States
(12) Patent Application Publication
(10) Pub. No.: US 2010/0069960 A1
(43) Pub. Date: Mar. 18, 2010

(54) Spinous Process Based Laminoplasty

(76) Inventor: Christopher D. Chaput, Temple, TX (US)

Correspondence Address: TROP, PRUNER & HU, P.C., 1616 S. VOSS ROAD, SUITE 750, HOUSTON, TX, 77057-2631, US

(21) Appl. No.: 12/561,580

(22) Filed: Sep. 17, 2009

Related U.S. Application Data
(60) Provisional application No. 61/192,285, filed on Sep. 17, 2008.
(60) Provisional application No. 61/195,996, filed on Oct. 14, 2008.

(51) Int. Cl. A61B 17/70

(52) U.S. Cl. 606/249; 606/248

(57) ABSTRACT

In one embodiment of the invention, a cervical implant may include a spinous process base coupled to a lateral mass base. During an open door laminoplasty, a surgeon may connect the lateral mass base to the right lateral mass and connect the spinous process base to the spinous process to fixedly retract the left lamina from the left lateral mass. As a result, no implant is needed to bridge or span the open door. Instead, the implant may span the hinged lamina. Consequently, no holes must be drilled in left lamina (open door side). If no holes must be drilled in left lamina, the surgeon lessens the risk of drilling a drill bit through left lamina and into spinal cord. Furthermore, no implant base or portion, screw, bolt, or fastener of any form needs to be affixed to inferior side of the left lamina, thereby avoiding irritation of the spinal cord by the implant.

This application claims priority to U.S. Provisional Patent Application No. 61/192,285 filed on Sep. 17, 2008 entitled SPINOUS PROCESS BASED LAMINOPLASTY and U.S. Provisional Patent Application No. 61/195,996 filed on Oct. 14, 2008 entitled SPINOUS PROCESS BASED LAMINOPLASTY.

### BACKGROUND

As discussed in U.S. Pat. No. 7,264,620 and illustrated in FIG. 1, a laminoplasty procedure may include performing an osteotomy in which a complete cut 102 is made through vertebra 101, approximately between the left lamina 103 and left lateral mass 110, such as the articular mass or facet portion thereof. A partial-depth cut 104 forming a “hinge” is made on the opposite lateral side, approximately between the right lamina 106 and right lateral mass 115. The left lamina 103, spinous process 120, and right lamina 106 are then hinged or pivoted about the partial cut 104 resulting in a “door” that pivots about “hinge” 104 to reveal an “open door” between the left lateral mass 110 and left lamina 103. The pivoting increases the cross-sectional size of the spinal canal to decompress the spinal cord 105 therein.

FIG. 2 illustrates a laminectomy wherein the left lamina, spinous process, and right lamina have been removed entirely and replaced with a cross member 123. Bone screw 132 may couple cross member 123 to left lamina 110 and bone screw 157 may couple cross member 123 to right lamina 115. Screws 132, 157 may be polyaxial screws as described more fully in United States patent application 2006/0064091. Use of such screws may allow flexibility in coupling cross member 123 to screws 132, 157. Furthermore, screws 132, 157 may accommodate rods 121, 122 that may couple (e.g., indirectly connect) to other cross bars and screws (not shown) set in a vertebra adjacent to vertebra 101. This adds spinal stabilization. With the spinal column stabilized, bone matter may be packed between vertebra 101 and adjacent vertebra to promote bone fusion between vertebrae.

FIG. 3 illustrates vertebra 101 after performance of an open-door laminoplasty. A cervical implant 130 spans the open door gap between left lateral mass 110 and left lamina 103. Cervical implant 130 couples to lateral mass 110 via a fastener 132 and to left lamina 103 via an additional fastener 131 using traditional techniques.

### BRIEF DESCRIPTION OF THE DRAWINGS

The accompanying drawings, together with the description of the invention, explain such various embodiments of the invention. In the drawings:

FIG. 1 illustrates a conventional method of spinous process based laminoplasty.

FIG. 2 illustrates a conventional method for performing a laminectomy.

FIG. 3 illustrates a conventional method for performing an open-door laminoplasty.

FIGS. 4-5, 7, 11 illustrate methods for performing open-door laminoplasty in various embodiments of the invention.

FIGS. 6, 8-10 illustrate embodiments of the invention.

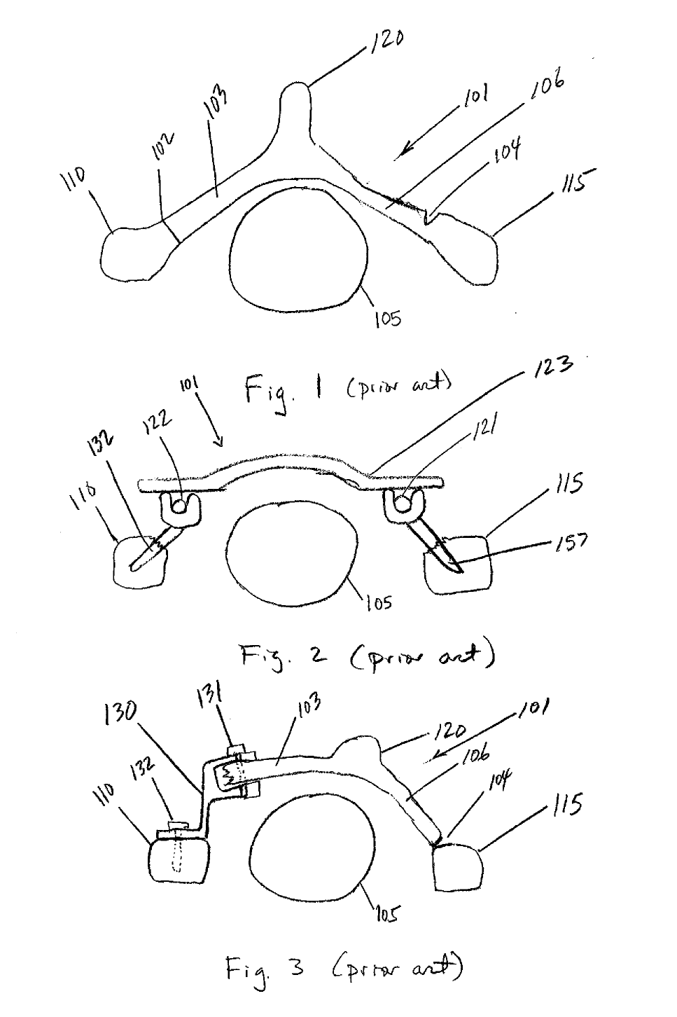

### DETAILED DESCRIPTION

The following description refers to the accompanying drawings. Among the various drawings the same reference numbers may be used to identify the same or similar elements. While the following description provides a thorough understanding of the various aspects of the claimed invention by setting forth specific details such as particular structures, architectures, interfaces, and techniques, such details are provided for purposes of explanation and should not be viewed as limiting. Moreover, those of skill in the art will, in light of the present disclosure, appreciate that various aspects of the invention claimed may be practiced in other examples or implementations that depart from these specific details. At certain junctures in the following disclosure, descriptions of known devices and methods have been omitted to avoid clouding the description of the present invention with unnecessary detail.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements co-operate or interact with each other, but they may or may not be in direct physical contact. As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner. References to “one embodiment”, “an embodiment”, “example embodiment”, “various embodiments”, etc., indicate that the embodiment(s) of the invention so described may include particular features, structures, or characteristics, but not every embodiment necessarily includes the particular features, structures, or characteristics. Further, some embodiments may have some, all, or none of the features described for other embodiments.

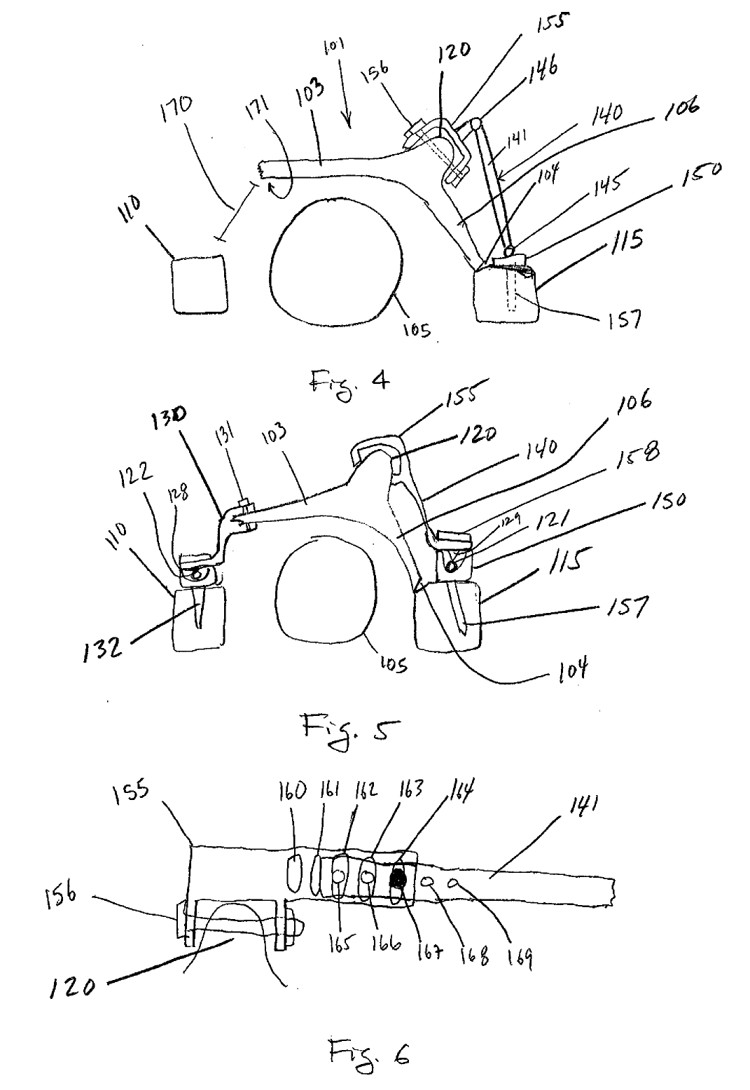

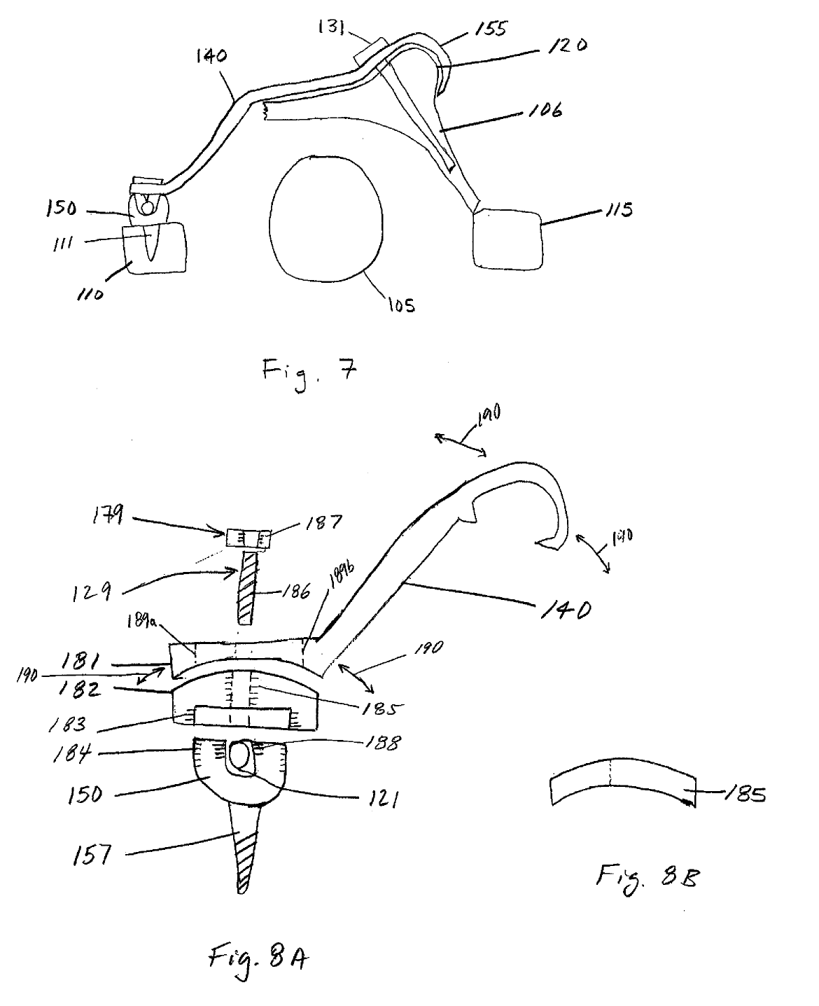

FIG. 4 illustrates one embodiment of an invention for performing spinous process based laminoplasty. Cervical vertebra 101 includes a left lateral mass 110 directly connected (not shown) to a left lamina 103, the left lamina 103 directly connected to a spinous process 120, the spinous process 120 directly connected to a right lamina 106, the right lamina 106 directly connected to a right lateral mass 115. A surgeon performs an osteotomy to disconnect the direct connection between the left lateral mass 110 and the left lamina 103. The surgeon then creates a groove or hinge 104 between the right lateral mass 115 and the right lamina 106. The surgeon pivots the left lamina 103, spinous process 120, and right lamina 106 about the hinge 104 to retract or withdraw the left lamina 103 from the left lateral mass 110 thereby creating open door 170. The surgeon then implants a cervical implant 140 into the patient. First cervical implant 140 includes a spinous process base 155 coupled to a lateral mass base 150 via a bridge or expanse portion. The surgeon directly connects the lateral mass base 150 to the right lateral mass 115 and directly connects the spinous process base 155 to the spinous process 120 to fixedly retract (i.e., provide a level of stability conducive to healing) the left lamina 103 from the left lateral mass 110. Thus, the laminar bridge spans a portion of hinged lamina 106 of the vertebra, but no portion of open door void 170 of the vertebra, to fixedly retract spinous process 120 from open door void 170 without any portion of the system spanning the open door void. As a result, no implant is needed to bridge or span open door 170. Consequently, no holes must be drilled in left lamina 103. If no holes must be drilled in left lamina 103, the surgeon lessens the risk of drilling a drill bit through left lamina 103 and into spinal cord 105. Furthermore, no implant base or portion, screw, bolt, or fastener of any form needs to be affixed to inferior side 171 of left lamina 103, thereby avoiding irritation of the spinal cord 105 by the implant.

Cervical implant 140 may include pivot members 145, 146 to provide flexibility in orienting the implant 140. In other words, pivot member 146 may allow flexibility in orienting span member 141 to base 155 and pivot member 145 may allow flexibility in orienting span member 141 to base 150. Any pivot member may be fixed at a particular orientation using methods such as, for example only, coupling a notched face of an axis in the pivot member against a complimentary notched face on a fastener which may be compressed against the pivot member using a bolt and nut. Of course other embodiments of the invention may include fewer (e.g., 0) or more (e.g., 3) pivot members. Base 155 may be coupled to spinous process 120 using a fastener (e.g., bone screw, bone anchor, monoaxial screw, polyaxial screw, bolts, hooks, crimping members, clamp, nail, suture, wire, rod, barbed member or any other apparatus for engaging or connecting to bone) 156. Base 150 may be coupled to lateral mass 115 using fastener 157.

FIG. 5 illustrates an embodiment of the invention for performing spinous process based laminoplasty. The cervical implant 140 that couples to the spinous process 120 and right lateral mass 115 is similar to the implant of FIG. 4. However, the implant differs in that the cervical implant 140 (FIG. 5) includes a spinous process base 155 that crimps, via one or more crimping arms or members, to spinous process 120. Furthermore, the lateral mass base 150 couples to the right lateral mass 115 using a polyaxial screw. A bridge or expanse couples lateral mass base 150 to spinous process base 155. As mentioned above, embodiments of polyaxial screws are described in United States Patent Application 2006/0064091. The polyaxial screw has a threaded shaft component 157 that couples to the lateral mass 115. In an embodiment, an optional cervical implant 130 may be used to span the open door of the open door laminoplasty. The implant 130 may include a polyaxial screw with a threaded shaft component 132 connected to left lateral mass 110. The implant 130 may couple to the left lamina 103 via a fastener (e.g., bone screw) 131. In some embodiments, the implant 130 may couple to the left lamina 103 via methodology that avoids using a translaminar fastener or fastener that resides between left lamina 103 and spinal cord 105. For example, implant 130 may use crimping or sutures affixed at, for example, spinous process 120 or a screw implanted in an intralaminar fashion in either lamina. In other embodiments, implant 130 may use a bracket affixed at and around the severed end of the open door lamina to stabilize the lamina, without using a translaminar pin driven through the bracket. For example, the severed end may wedge or sit within the bracket and maintain its location due to, in part, the retractive forces of implant 140.

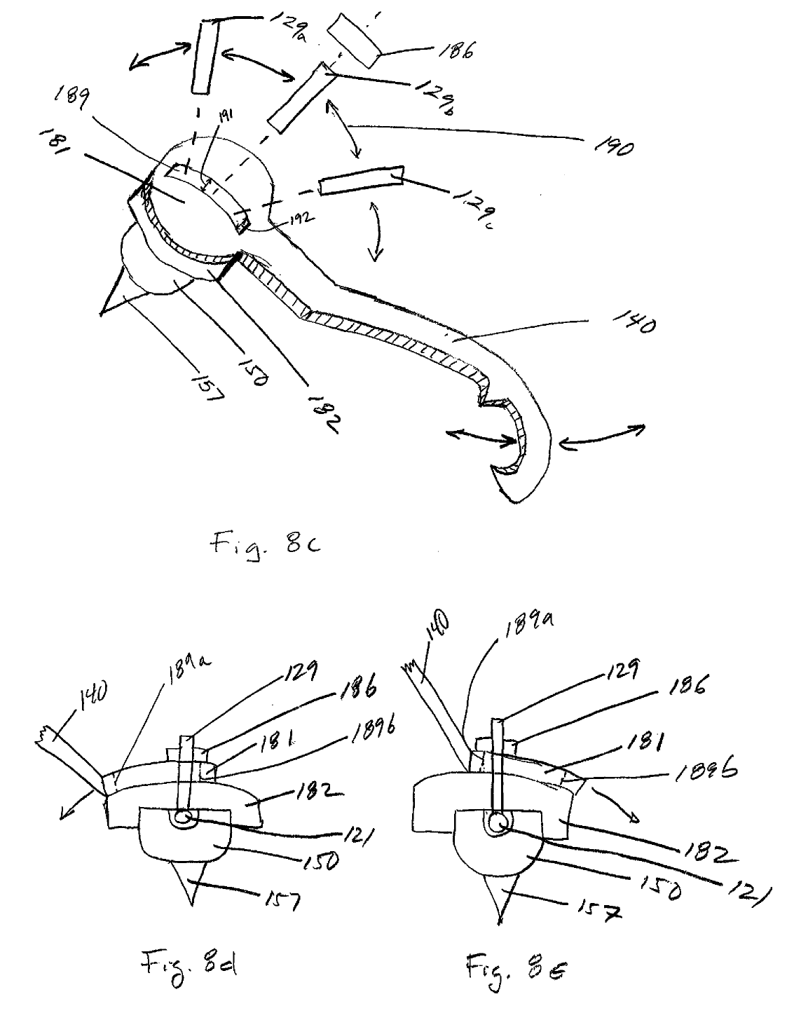

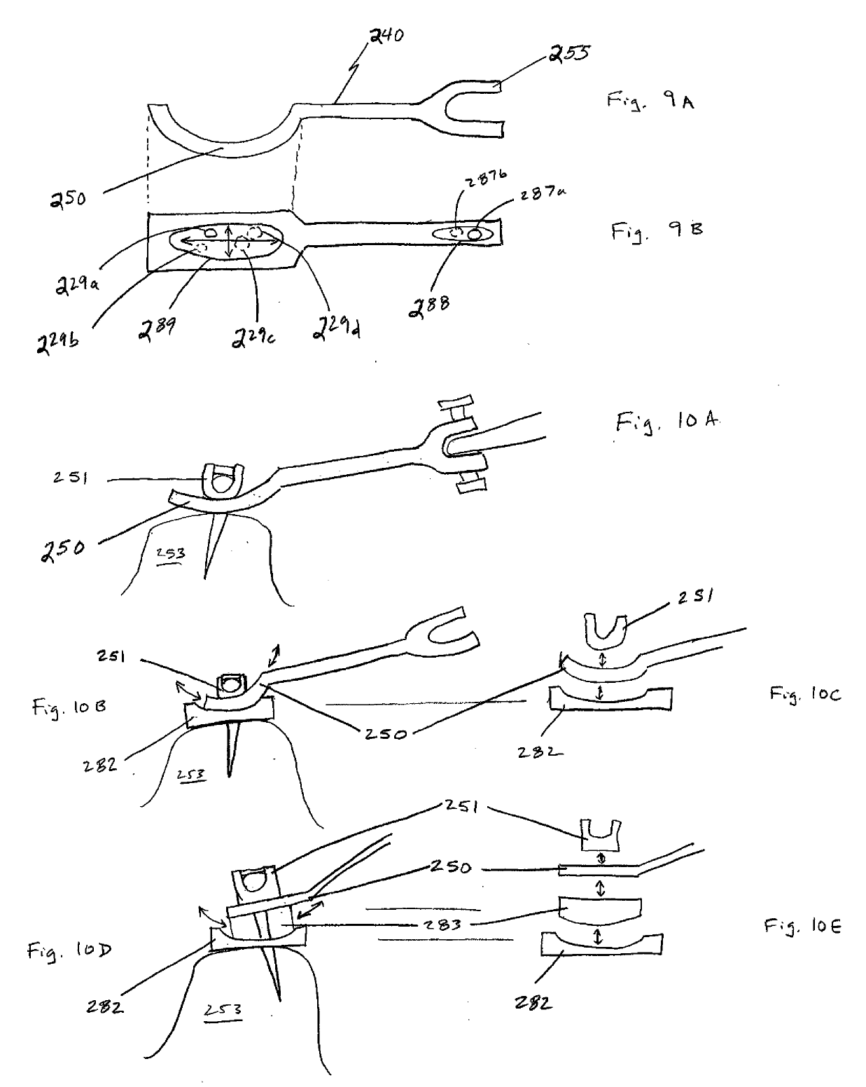

FIG. 6 illustrates another manner in which flexibility is included in various embodiments of the invention. Spinous process 120 is coupled to base 155 using screw 156. Base 155 includes a series of slots 160, 161, 162, 163, 164. Base 155 may include a conduit portion with an inner diameter that is greater than the outer diameter of span member 141. Consequently, span member 141 may be received within base member 155. Span member 141 may include a series of apertures 165, 166, 167, 168, 169. The surgeon may then align any one aperture 165, 166, 167, 168, 169 with any one slot 160, 161, 162, 163, 164 to variably control the length of the base 155/span member 141 combination. Any aperture 165, 166, 167, 168, 169 aligned with any slot 160, 161, 162, 163, 164 may be coupled together using, for example only, a fastener such as a screw or suture.

In one embodiment of the invention, a surgeon may locate a second cervical vertebra adjacent to a vertebra upon which a laminoplasty has already been performed (e.g., see FIG. 5). The second cervical vertebra (not shown) may include a second left lateral mass directly connected to a second left lamina, the second left lamina directly connected to a second spinous process, the second spinous process directly connected to a second right lamina, the second right lamina directly connected to a second right lateral mass. After disconnecting the direct connection between the second left lateral mass and the second left lamina, a surgeon may create a second groove between the second right lateral mass and the second right lamina as described above. After pivoting the second right lamina and second spinous process about the second groove to retract the second left lamina from the second left lateral mass, the surgeon may implant a second cervical implant into a patient, the second cervical implant including a second spinous process base coupled to a second lateral mass base as described above in regards to FIG. 5. The surgeon may then directly connect (i.e., without intervening material) the second lateral mass base to the second right lateral mass and directly connect the second spinous process base to the second spinous process to fixedly retract the second left lamina from the second left lateral mass.

Thus, in the embodiment described immediately above, two adjacent vertebrae may be configured as seen in FIG. 4 or FIG. 5 (with or without the cervical implant spanning the open door). The adjacent vertebrae may then be stabilized to one another. In one embodiment of the invention, the surgeon may employ a fixation element (e.g., rod, plate, cable, tether or any other apparatus to fixate bone) 121 to couple the first cervical implant (e.g., see FIG. 5) to the second cervical implant (not shown). Coupling cervical implants/vertebrae adjacent to or near each other adds stability to the implant system/vertebrae. A set screw 129 may set the fixation element 121 (e.g., rod) in place. If implants spanning the open door are used, a second fixation element 122 (e.g., rod) may additionally couple the vertebrae together. Set screw 128 may set the fixation element 122 in place on the first vertebra and additional set screw (not shown) may set the fixation element 122 in place in a second implant coupled to a second vertebra (not shown). Additional bone matter may then be coupled to one or both of the stabilized vertebrae to permit bone fusion of the vertebrae. By coupling laminoplasty with stabilization (e.g., via fixation element 121 and/or 122) there is more bone surface area available to aide fusion than would be the case if a laminectomy were performed on the vertebrae (i.e., where the lamina are removed as illustrated in FIG. 2).

While the above example regarding stabilization and fusion has discussed adjacent vertebrae, the invention is not so limited and may stabilize any appropriate number of adjacent or nonadjacent vertebrae to one another.

In another embodiment of the invention, an implant spans the open door and directly connects, for example only, the left lateral mass to the spinous process to fixedly keep the open door retracted. Such an embodiment would still avoid drilling holes in the left lamina that increase the chances of undesirable contact with the spinal cord. In such an embodiment of the invention, the left lamina may or may not be removed. In other embodiments, a fixation element (e.g., rod) used to couple two vertebrae may couple implant bases that are connected to spinous processes (i.e., instead of or in addition to coupling bases connected to lateral masses).

FIG. 7 illustrates an embodiment of the invention for performing spinous process based laminoplasty. The cervical implant 140 couples the spinous process 120 and left lateral mass 110. Implant 140 includes a spinous process base 155 that crimps to the spinous process 120. Furthermore, the lateral mass base 150 couples to the left lateral mass 110 using a polyaxial screw 111. Spinous process base 155 couples to spinous process 120 using a screw 131 implanted in an intralaminar fashion. Intralaminar fixation is thus an option in addition to, for example, translaminar fixation (see, e.g., screw 131 in FIG. 5).

FIG. 11 illustrates an embodiment of the invention for performing spinous process based laminoplasty. The cervical implant 140 couples the spinous process 120 and left lateral mass 110 and right lateral mass 115. Lateral mass base 150 couples to left lateral mass 110 using a polyaxial screw 111 and lateral mass base 151 couples to right lateral mass 115 using a polyaxial screw 112. Spinous process base 155 couples to spinous process 120 using a screw 131 implanted in an intralaminar fashion, although other embodiments may include other forms of fixation (see, e.g., translaminar fixation screw 131 in FIG. 5). Implant 140 may be somewhat flexible and/or include various pivots, as described herein, to configure implant 140 in various configurations to aid positioning implant 140 in relation to various anatomical features.

FIG. 8a illustrates another embodiment of the invention. Base 150 may be coupled to a lateral mass using fastener 157. Base 150 may include a threaded portion 184 to couple to a second threaded portion 183 of convex cap 182. Cap or spacer 182 may couple to the non-linear surface comprising concave base 181 using, for example, set screw 129 that sets rod 121 using reciprocating threads 185, 186, 188. Base 181 may couple to set screw 129 using nut 179 and threads 186, 187. FIG. 8b illustrates a spacer or washer 185 that may be located between, for example, cap 182 and base 181 for greater flexibility in using device 140. In other embodiments, cap 182 need not be non-linear (e.g., convex) and base 181 need not be non-linear (e.g., concave). FIG. 8c illustrates a rotational aspect of the invention. For example, base 181 includes a slot 189 that receives set screw 129 (depicted in various positions as 129a, 129b, 129c). As a result, base 181 may slide or pivot (e.g., direction 190) in relation to screw 129 and cap 182. Slot 189 may include, for example, guide rails and reciprocating slots to control freedom of movement between base 181 and cap 182. Slot 189 may include a diameter 191 that is equal to diameter 192. However, in other embodiments diameter 191 may be greater than diameter 192 thereby creating, for example, an ovular slot that allows freedom for placing base 181.

FIGS. 8d and 8e show differently oriented assemblies of an embodiment of the invention. FIG. 8d illustrates base 181 slid to the left so that screw 129 is located near the right edge 189b of slot 189 and away from left edge 189a. Base 181 includes a non-linear surface that is complementary to a non-linear surface included in cap 182. The non-linear surfaces may traverse (e.g., slide, pivot, move, rotate) across one another to provide flexibility in coupling the device to the vertebra. For example, the non-linear surfaces may be slidably coupled to one another. FIG. 8e illustrates base 181 slid to the right so that screw 129 is located near the left edge 189a of slot 189 and away from right edge 189b. Thus, FIGS. 8a-8e illustrate an embodiment of the invention that gives the surgeon greater flexibility in positioning device 140 in relation to the patient's specific anatomical features. The embodiment of FIGS. 8a, 8b, 8c, 8d, and 8e may or may not be used in conjunction with other embodiments of the invention described herein (e.g., FIGS. 4, 5, 7) and may or may not be used in conjunction with spinous process based laminoplasty. For example, fixation element 121 (e.g., rod) or polyaxial screw 150 are not required in every embodiment of the invention. Other embodiments including slot 189 (but not polyaxial screws and/or stabilization rods) would still allow greater flexibility using traditional bone screws that could include a cap and base with the slot as described above.

FIGS. 9A and 9B are another embodiment of the invention. Base 205 may be rounded and may include an ovular void 289. Void 289 may allowed various placement locations 229a-d for pin 229. Similarly, void 288 allows various placement locations 287a-b for pin 287.

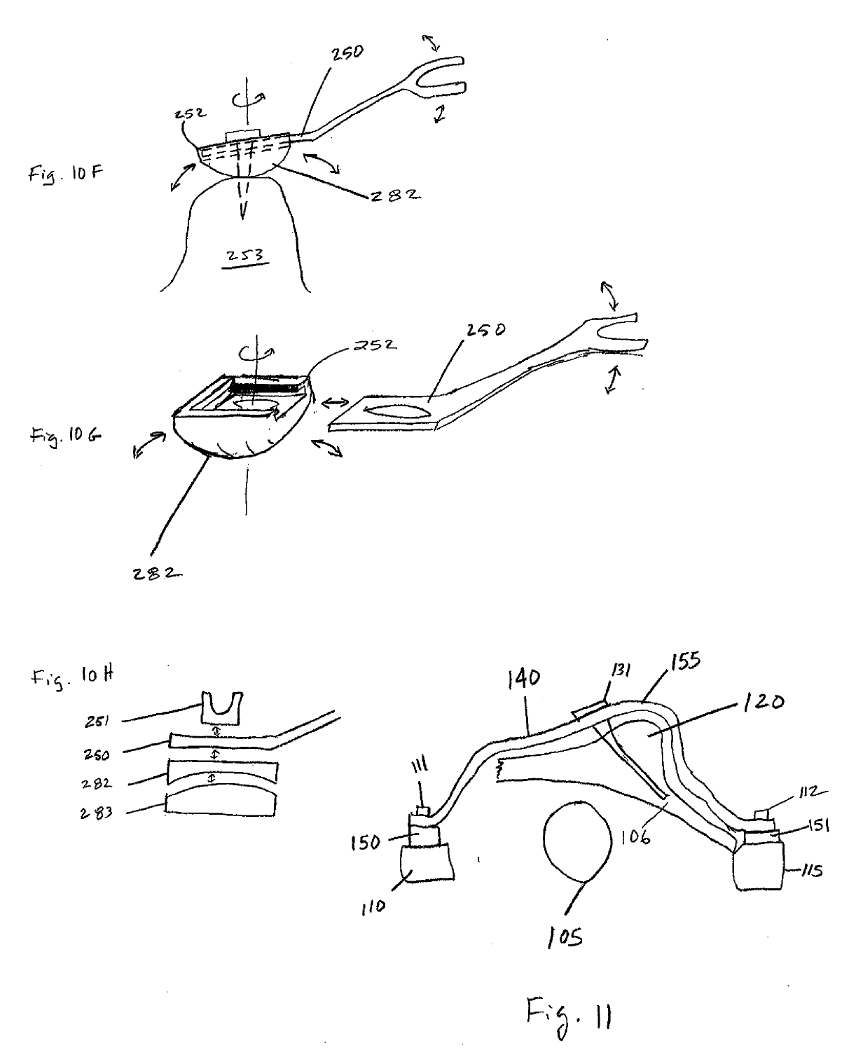

FIG. 10A shows an embodiment of the invention whereby nonlinear (e.g., cupped) base 250 may be coupled to bone 253 in a variety of positions and angles of rotation in numerous planes. Polyaxial pin 251 may set base 250 in a final orientation.

FIGS. 10B and 10C show an embodiment of the invention whereby nonlinear base 250 nests with nonlinear base 282. Pin 251 may in turn nest to base 250. For example, complementary surfaces including equal radii of curvature may allow for snug fits between items 251, 250, 282 while still allowing for sliding between those items. This provides more flexibility to the surgeon in locating the implant.

FIGS. 10D and 10E show an embodiment of the invention whereby nonlinear washer 282 nests with nonlinear spacer 283. Spacer 283 nests with base 250. Pin 251 may nest to base 250. Base 250 may be a “traditional” or conventional base used in the prior art that gains flexibility by coupling with washer 282 and/or spacer 283. Moving the base 250 closer to bone 253 (and/or driving screw or pin through washer and/or spacer and/or base) may provide clinical benefits (e.g., structural integrity of implant/bone combination).

FIGS. 10F and 10G show an embodiment of the invention whereby nonlinear washer 282 connects directly to bone 252. Washer 282 may include slots, rails, and guidance mechanisms 252 to better receive base 250. The underside of washer 282 may resemble an inverted dome, thereby facilitating freedom of motion in multiple planes of rotation (e.g., medial-lateral and cephalad-caudad). Washer 282 may include a void or hole with, for example, an ovular cross-section to allow translation in multiple directions on the horizontal plane before setting the washer 282 and plate 250 assembly with a bone screw. As made evident above, base 250 may be conventional but gain flexibility in orientation by coupling to washer 282.

The above embodiments may be arranged in various configurations using some or all components (e.g., spacers, washers). For example, washer 282 and spacer 283 may be arranged as shown in FIG. 10H. This nesting arrangement, like that of FIG. 10E, allows polyaxial screw 251 to couple with base 250 without torquing plate 250.

Other arrangements included within the scope of the invention. For example, the arrangement of FIG. 10H could be modified so that polyaxial screw component 251 connects on top of washer 282, which connects to the top of spacer 283, which connects to the top of base 250, which connects to bone. This configuration allows greater freedom for implanting screw 251 (or even a conventional, non-polyaxial screw) at a variety of angles in relation to the bone. Also, the bottom of screw component 251 could even be made to have a complementary radius of curvature so that it nests directly with a curved surface of spacer 283, thereby allowing the surgeon to determine whether washer 282 is needed. In addition, base 250 might have a curved upper portion that nests directly with a curved bottom portion of base 282. Furthermore, base 250 might even have a curved upper portion that nests directly with a complementary curved bottom portion of screw component 251, thereby reducing the number of parts in the assembly while still providing numerous degrees of freedom for implanting the device. Still other such arrangements, while not specifically addressed herein, are encompassed within the scope of the invention.

The above embodiments (e.g., FIGS. 9-10) may be used with conventional or nonconventional bases, conventional screws/pens or nonconventional screws/pins (e.g., polyaxial screws/pins), and with or without fusion. Furthermore, the embodiments may be used on the open door or the hinge side of laminoplasty configurations. The above embodiments provide more degrees of freedom to orient conventional laminoplasty bases.

In addition and more generally, the embodiments described above are not limited to implementation with cervical vertebrae but may be implemented on, without limitation, thoracic and lumbar vertebrae with modifications to, for example, spinous process base 155 (FIG. 4) to accommodate variously configured processes. Also, in certain embodiments above, full or partial cuts are made “between” a lateral mass and lamina but it should be understood that surgeons will vary the exact location of cuts according to the needs of the patient and general concepts of fixed retraction apply despite variances in cut locations. Also, while some of the above embodiments describe creating open door 170 between left lateral mass 110 left lamina 103, the invention is not so limited and is applicable in instances wherein, for example, the open door is formed between the right lamina and right lateral mass.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. An orthopedic fixation system comprising:
a lateral mass base configured to couple to a lateral mass of a vertebra;
a spinous process base configured to couple to a spinous process of the vertebra;
a laminar bridge coupling the spinous process base to the lateral mass base;
wherein when the system is used during an open door laminoplasty of the vertebra the laminar bridge spans a portion of a hinged lamina of the vertebra, but no portion of an open door void of the vertebra, to fixedly retract the spinous process from the open door void without any portion of the system spanning the open door void.

2. The system of claim 1 including:
a second lateral mass base configured to couple to a lateral mass of a second vertebra;
a second spinous process base configured to couple to a spinous process of the second vertebra;
a second laminar bridge coupling the second spinous process base to the second lateral mass base;
a rod;
wherein when the system is used during an open door laminoplasty of the second vertebra (a) the second laminar bridge spans a portion of a hinged lamina of the second vertebra, but no portion of an open door void of the second vertebra, to fixedly retract the second spinous process from the open door void of the second vertebra without any portion of the system spanning the open door void of the second vertebra; and (b) the rod couples the lateral mass base to the second lateral mass base to fixate the vertebra to the second vertebra.

3. The system of claim 1, wherein the spinous process base couples to first and second crimping arms, the crimping arms to crimp to the spinous process to fixedly retract the spinous process from the open door void when the system is used during the open door laminoplasty of the vertebra.

4. The system of claim 1, wherein the laminar bridge couples to the spinous process base via a first pivot and to the lateral mass base via a second pivot.

5. The system of claim 1 including a spacer that comprises a first nonlinear surface; wherein (a) the lateral mass base includes a second nonlinear surface that is complementary to the first upper nonlinear surface, and (b) when the system is used during the open door laminoplasty of the vertebra the spacer couples to the lateral mass base allowing the first nonlinear surface to traverse across the second nonlinear surface.

6. The system of claim 1 including:
a spacer that includes a first nonlinear surface; and
a screw including a second nonlinear surface that is complementary to the first nonlinear surface;
wherein when the system is used during the open door laminoplasty of the vertebra the spacer couples to the screw and the lateral mass base allowing the first nonlinear surface to traverse across the second nonlinear surface.

7. The system of claim 1 including a screw that includes a first nonlinear surface; wherein (a) the lateral mass base includes a second nonlinear surface that is complementary to the first nonlinear surface and (b) when the system is used during the open door laminoplasty of the vertebra the screw couples to the lateral mass base allowing the first nonlinear surface to traverse across the second nonlinear surface.

8. The system of claim 1 including a screw; wherein (a) the lateral mass base includes a first nonlinear surface, and (b) when the system is used during the open door laminoplasty of the vertebra the first nonlinear surface pivotally and directly connects to the lateral mass of the vertebra and fixates to lateral mass of the vertebra via the screw.

9. The system of claim 1 including a screw, a rod, and a spacer that includes a first nonlinear surface with a first radius of curvature; wherein (a) the lateral mass base includes a second nonlinear surface with a second radius of curvature that are respectively complementary to the first nonlinear surface and the first radius of curvature, and (b) when the system is used during the open door laminoplasty of the vertebra the rod couples the lateral mass base to another fixation device, the screw couples the lateral mass base to the lateral mass of the vertebra, and the spacer couples to the lateral mass base allowing the first nonlinear surface to traverse across the second nonlinear surface.

10. An orthopedic fixation system comprising:
a first lateral mass base configured to couple to a first lateral mass of a vertebra;
a first spinous process base configured to couple to a spinous process of the vertebra;
a first laminar bridge coupling the spinous process base to the lateral mass base;
a second lateral mass base configured to couple to a second lateral mass of the vertebra;
a second spinous process base configured to couple to the spinous process;
a second laminar bridge coupling the second spinous process base to the second lateral mass base;
wherein when the system is used during an open door laminoplasty of the vertebra (a) the first and second lateral mass bases respectively couple to the first and second lateral masses via bone screws, (b) the first and second spinous process bases couple to the spinous process, (c) the first laminar bridge spans a portion of a hinged lamina of the vertebra, but no portion of an open door void of the vertebra, to fixedly retract the spinous process from the open door void, and (d) the second laminar bridge spans a portion of the open door void of the vertebra to fixedly retract the spinous process from the open door void.

11. The system of claim 10, wherein the second spinous process base includes first and second crimping arms to fixatedly crimp to a portion of the vertebra when the system is used during an open door laminoplasty of the vertebra.

12. The system of claim 10, wherein the second spinous process base is configured to couple to the vertebra without using translaminar screws when the system is used during an open door laminoplasty of the vertebra.

13. An orthopedic fixation system comprising:
a lateral mass base coupled to a lateral mass of a vertebra that has been subjected to an open door laminoplasty;
a spinous process base coupled to a spinous process of the vertebra;
a laminar bridge coupling the spinous process base to the lateral mass base;
wherein the laminar bridge spans a portion of a hinged lamina of the vertebra, but no portion of an open door void of the vertebra, and fixedly retracts the spinous process from the open door void.

14. The system of claim 13 including:
a second lateral mass base coupled to a lateral mass of a second vertebra that has been subjected to an open door laminoplasty;
a second spinous process base coupled to a spinous process of the second vertebra;
a second laminar bridge coupling the second spinous process base to the second lateral mass base;
a rod;
wherein (a) the second laminar bridge spans a portion of a hinged lamina of the second vertebra, but no portion of an open door void of the second vertebra, and fixedly retracts the second spinous process from the open door void of the second vertebra; and (b) the rod couples the lateral mass base to the second lateral mass base and fixates the vertebra to the second vertebra.

15. The system of claim 13, wherein the spinous process base couples to first and second crimping arms, the crimping arms to crimp to the spinous process and fixedly retract the spinous process from the open door void.

16. The system of claim 13 including:
a first nonlinear surface with a first radius of curvature coupled to the lateral mass base; and
a second nonlinear surface with a second radius of curvature, coupled to the lateral mass base, respectively complementary to the first nonlinear surface and the first radius of curvature;
wherein the first nonlinear surface slidably and directly connects to the second nonlinear surface to fixate the system at first angle in relation to the first lamina.

17. An orthopedic fixation system comprising:
a lateral mass base coupled to a lateral mass of a vertebra;
a spinous process base coupled to a spinous process of the vertebra; and
a laminar bridge;
wherein the laminar bridge couples the spinous process base to the lateral mass base.

18. The system of claim 17, wherein the laminar bridge couples the spinous process base to the lateral mass base to fixedly retract the spinous process from an open door void.

19. The system of claim 18, wherein the laminar bridge spans a portion of an open door void.

20. The system of claim 19 including:
a second lateral mass base coupled to a second lateral mass of a vertebra; and
a second laminar bridge;
wherein the second laminar bridge couples the spinous process base to the second lateral mass base and spans a portion of a hinged lamina.
